(19) United States
(12) Patent Application Publication
(10) Pub. No.: US 2013/0131537 A1
(43) Pub. Date: May 23, 2013

(54) TONG REN BRAINWAVE ENTRAINMENT

(71) Applicant: Thomas Tam, Quincy, MA (US)

(72) Inventor: Thomas Tam, Quincy, MA (US)

(21) Appl. No.: 13/671,990

(22) Filed: Nov. 8, 2012

Related U.S. Application Data
(60) Provisional application No. 61/556,949, filed on Nov. 8, 2011.

(51) Int. Cl. A61B 5/00; A61B 7/00; A61B 5/0476

(52) U.S. Cl. CPC A61B 5/4854; A61B 5/0476; A61B 7/00; A61B 5/6803; A61B 5/6831
USPC 600/544

(57) ABSTRACT

A system and method for creating a Tong Ren Brainwave Entrainment (TRBE) signal. Electroencephalography (EEG) signals are recorded from a Tong Ren practitioner during a session. Practitioner EEG signals are processed and appended to produce TRBE audio compositions for select applications according to the type of Tong Ren session.

### RELATED APPLICATION

This application claims the benefit of U.S. Provisional Application No. 61/556949 filed Nov. 8, 2011, incorporated herein by reference in its entirety for all purposes.

### COPYRIGHT NOTICE

A portion of the disclosure of this patent document contains material that is subject to copyright protection. The copyright owner has no objection to the facsimile reproduction by anyone of the patent document or the patent disclosure, as it appears in a published Patent and Trademark Office patent file or record, but otherwise reserves all copyrights whatsoever.

### FIELD OF THE INVENTION

The invention relates to a system and method for creating a Tong Ren Brainwave Entrainment (TRBE) signal. Electroencephalography (EEG) signals are recorded from a Tong Ren practitioner during a session. Practitioner EEG signals are processed and appended to produce TRBE audio compositions for select applications according to the type of Tong Ren session.

### BACKGROUND OF THE INVENTION

Electrical activity in the brain produces detectable brain wave signals. The frequencies are associated with changes in mental state and cognitive ability. During daily waking life, a normal human brain functions at frequencies ranging from 13 to 25 Hertz or cycles per second, called the Beta state. During times of rest or daydreaming, brainwave cycles drop to 8 to 12 cycles per second, or the Alpha state. When sleeping, Theta and Delta states are dominant, ranging from 4 to 7 and 0.2 to 4 cycles per second respectively.

### Brainwave Entrainment

Brain waves can be altered by presenting stimuli through the ears or eyes at repetition rates equal to the desired brain wave frequency. This is called brainwave entrainment. For example, if a 5 beat per second audio loop is played to a subject over a period, the subject's brain will entrain to 5 cycles per second, or brain activity at the 5 Hz frequency will increase. Because brain waves relate to mood and attention levels, researchers believe that stimulating frequencies can result in a variety of effects.

Brainwave entrainment is also known as brainwave synchronization. This is any practice that aims to cause brainwave frequencies to fall into step with periodic stimulus having a frequency corresponding to the intended brain-state, usually attempted with the use of specialized software. It purportedly depends upon a “frequency following” response on the assumption that the human brain has a tendency to change its dominant electroencephalogram (EEG) frequency towards the frequency of a dominant external stimulus. Such a stimulus is often aural, as in the case of binaural or monaural beats and isochronic tones or else visual, as with a dream machine, a combination of the two with a mind machine, or even electromagnetic radiation.

Previous inventions have used flashing lights or pulsing tones to produce brain wave entrainment, as shown by U.S. Pat. Nos. 4,315,502 and 5,289,438. However, pure tones and tone pulses can be quite invasive. Previous inventions have endeavored to solve this problem by masking over the entrainment with white noise, such as in U.S. Pat. No. 5,213,562 or music. Unfortunately, masking the brain wave entraining sounds of an audio composition can decrease the effectiveness of the brain stimulation.

Music alone has been used to affect the brain such as in U.S. Pat. No. 5,586,967, which describes a method and apparatus for inducing enhanced states of learning by presenting musical sounds in ascending and descending crescendos The tempo of music stimulating brain waves is limited, since brain wave frequencies range as high as 60 cycles per second.

Other methods have endeavored to tackle this problem by applying modulations non-specifically to entire pieces of music. Modulations alter sound to create a pattern of beats where none existed before. The basic problem remains, since the only way to create subtle, non-invasive audio entrainment in these approaches is to decrease the intensity of the modulation, thus decreasing the overall effectiveness of the brain wave entrainment.

### Tong Ren

Tong Ren practice derives from the concepts of the collective unconsciousness and brainwave entrainment. The collective unconscious is a term of analytical psychology, coined by Carl Jung. It is proposed to be a part of the unconscious mind, expressed in humans and all life forms with nervous systems, and describes how the structure of the psyche autonomously organizes experience. Jung distinguished the collective unconscious from the personal unconscious, in that the personal unconscious is a personal reservoir of experience unique to each individual, while the collective unconscious collects and organizes those personal experiences in a similar way with each member of a particular species. See, for example, Tong Ren Healing Classes published by the OCI Healing Research Foundation (Appendix A), Tong Ren Therapy Cross Reference (Appendix B), and Tong Ren Healing Method: A Survey Study published by SagePub, http://chp.sagepub.com/, also published online before print Dec. 23, 2008, doi: 10.1177/1533210108329265 Journal of Evidence-Based Complementary & Alternative Medicine January 2009 vol. 14 no. 1 19-35, whose contents are herein incorporated by reference.

Tong Ren has been developed by Tom Tam, a practicing acupuncturist since 1981, and is a form of energy therapy for restoring health and vitality. In Mandarin, Tong Ren translates to ‘Bronze Man’, and refers to teachers of acupuncture from the Sung Dynasty (1023 AD) who tested their students using bronze figures. Tong Ren is based on an understanding that disease is related to interruptions, or blockages, in the body's natural flow of blood, hormones, neural bioelectricity, or chi. Tong Ren seeks to remove these blockages, restoring the body's natural ability to heal itself, even when illnesses are chronic, debilitating, or otherwise untreatable.

Tong Ren combines western knowledge of anatomy and physiology with the ancient principle of “chi,” or life force energy. While similar to Chi Gong therapy and acupuncture in that it purports to affect the flow of chi that is hypothesized to sustain life, it draws from western science and traces the flow of chi along the physiological pathways of the endocrine, circulatory, central, and peripheral nervous systems. As mentioned, it draws also from the Jungian theory of the collective unconscious, to access energy from this universal source and direct it to the patient. No physical contact is involved or necessary and Tong Ren is often practiced as distance healing.

The National Institutes of Health (NIH) National Center for Complementary Medicine and Alternative Medicine has described energy medicine as a domain in complementary and alternative medicine that is deserving of further research. Tong Ren is an energy medicine that belongs in the category of putative medicine, also referred to as subtle energy or biofields, because the energy can be difficult to measure accurately. The Tong Ren healing modality has become increasingly popular among cancer patients and others with serious, chronic and life threatening disease.

What is needed is a method to produce a recorded brainwave entrainment signal for producing the effects of Tong Ren in individuals without requiring a direct session.

### SUMMARY OF THE INVENTION

Embodiments of the invention include a system for creating a Tong Ren brainwave entrainment electroacoustic (TRBE) composition comprising the steps of recording electroencephalogram (EEG) signals of a practitioner during a session; the recording comprising at least one channel of the EEG signals of the practitioner; removing DC offset for each of the at least one channel of the EEG signals of the practitioner; scaling each of the at least one channel of the EEG signals of the practitioner; generating, for each of the at least one channel of the EEG signals of the practitioner a sound wave recording; saving the at least one sound wave recording; selecting at least one of the saved at least one sound wave recordings; performing, on at least one of the selected sound wave recordings, additional audio signal editing comprising DC offset correction; and performing, on at least one of the selected sound wave recordings, audio signal editing comprising volume amplification; whereby the TRBE electroacoustic composition corresponding to the at least one specific ailment is created. In another embodiment, the recording of at least one channel of the EEG signals of the practitioner comprises fourteen channels. For a further embodiment, the session further comprises the practitioner rhythmically tapping a Tong Ren hammer on a Tong Ren Human figurine. In other embodiments, the at least one sound wave recording is in digital audio file format. An additional embodiment further comprises selecting a recorded session composition; connecting a Tong Ren Wave transducer to audio output of an audio/music player; activating the Tong Ren Wave transducer by playing the selected Tong Ren Wave file from the audio/music player; and placing the Tong Ren Wave transducer against the surface of a body for a specific area to be stimulated. Yet another embodiment comprises bone conduction of the transmission. In continuing embodiments, the transmission comprises about 25 kilohertz at about 10 milliwatts power; and in other embodiments the transmission is between about 4 Hz to about 200 kHz. Subsequent embodiments include that the step of placing the Tong Ren Wave transducer is accomplished remotely over communication lines from source of the transmission. Ongoing embodiments further comprise a low frequency Tong Ren wave transducer device; a high frequency Tong Ren wave transducer device; a Tong Ren wave eyeglasses device; a Tong Ren wave head band device; and a Tong Ren wave belt device.

Another embodiment of the invention includes a method for creating a Tong Ren brainwave entrainment (TRBE) electroacoustic composition comprising the steps of recording electroencephalogram (EEG) signals of a practitioner during a session; the session comprising the practitioner selecting at least one meridian point on a Tong Ren Human figurine corresponding to a corresponding location where a subject has bioelectric blockages common to the subject's corresponding at least one specific ailment; the session further comprising the practitioner rhythmically tapping a Tong Ren hammer on the Tong Ren Human figurine; the recording comprising at least one channel of the EEC signals of the practitioner; removing the entire DC offset for each of the at least one channel of the EEG signals of the practitioner; scaling each of the at least one channel of the EEG signals of the practitioner; generating, for each of the at least one channel of the EEG signals of the practitioner, a sound wave recording, wherein the step of generating the sound wave recording comprises sound wave conversion software; saving the at least one sound wave recording into digital audio file format; selecting at least one of the saved at least one sound wave digital audio file format recordings; performing, on at least one of the selected sound wave digital audio file format recordings, additional audio signal editing comprising DC offset correction; and performing, on at least one of the selected sound wave digital audio file format recordings, audio signal editing comprising volume amplification; whereby the TRBE electroacoustic composition corresponding to the at least one specific ailment is created. Embodiments provide that on at least one of the selected sound wave digital audio file format recordings, signal filtering comprising low pass, band pass, band stop, and high pass filtering is accomplished. Other embodiments include mixing a plurality of signals. For further embodiments, the plurality of signals comprises the selected sound file, other non brainwave sound tracks, at least one other brain wave channel, and at least one additional sound file. An ensuing embodiment further comprises simultaneously activating a headphone audio player to listen to the Tong Ren Wave.

Yet another embodiment of the invention includes a system for Tong Ren brainwave entrainment (TRBE) comprising recording and processing electroencephalogram (EEG) signals of a practitioner during a session; selecting a recorded session; connecting a Tong Ren Wave transducer to audio output of an audio/music player; activating the Tong Ren Wave transducer by playing the selected Tong Ren Wave file from the audio/music player; placing the Tong Ren Wave transducer against the surface of the body for a specific area to be stimulated.

The features and advantages described herein are not all-inclusive and, in particular, many additional features and advantages will be apparent to one of ordinary skill in the art in view of the drawings, specification, and claims. Moreover, it should be noted that the language used in the specification has been principally selected for readability and instructional purposes, and not to limit the scope of the inventive subject matter.

### BRIEF DESCRIPTION OF THE DRAWINGS

FIG. 1 depicts diagrams of a five step Tong Ren brainwave recording process 1A and overall application 1B configured in accordance with an embodiment.

FIG. 2 depicts a Tong Ren figure and Tong Ren hammer.

FIG. 3 displays practitioner with neuroheadset configured in accordance with an embodiment.

FIG. 4 depicts the sixteen contact point locations for the neuroheadset configured in accordance with an embodiment.

FIG. 5 depicts a screen of a TestBench® application for brainwave recording configured in accordance with an embodiment of the invention.

FIG. 6 depicts a recorded EPOC EEG data set segment for a single channel configured in accordance with an embodiment.

FIG. 7 depicts a sound wave waveform generated for the EEG data segment configured in accordance with an embodiment.

FIG. 8 depicts a sound wave waveform shown in units of decibels configured in accordance with an embodiment.

FIG. 9 depicts EEG channel data sets shown in Microsoft® Excel® spreadsheet format configured in accordance with an embodiment. Microsoft and Excel are trademarks of the Microsoft group of companies.

FIG. 10 depicts a graph of DC offset correction in accordance with an embodiment.

FIG. 11 depicts a graph showing a lower track with higher amplitude compared to the upper track after amplification was applied in accordance with an embodiment.

FIG. 12 depicts a screen shot of Audacity® WAVE to MP3 Export options configured in accordance with an embodiment.

FIG. 13 depicts a graph of three separate sound waves—(1) Arthritis AF3 channel brainwave sound wave with insertions of (2) introduction and (3) ending sound waves configured in accordance with an embodiment.

FIG. 114 depicts a graph of the three sound waves shown in FIG. 13 mixed into a single sound track configured in accordance with an embodiment.

FIG. 15 depicts graphs 15A of five separate sound waves Arthritis brainwave channels AF3, F7, P8, introduction and ending sound waves; 15B mixing with 4 Hz sine wave with TRBE audio file; and 15C mixing with pulse wave with TRBE audio file configured in accordance with an embodiment.

FIG. 16 depicts a graph of five sound waves shown in FIG. 15 mixed into a single sound track configured in accordance with an embodiment.

FIG. 17 depicts a display of Tong Ren Brainwave MP3 file configured in accordance with an embodiment.

FIG. 18 is a high level flow chart of operation of a method of Tong Ren Brainwave Entrainment configured in accordance with an embodiment.

FIG. 19 is a flow chart of the recording creation operation of a method of Tong Ren Brainwave Entrainment configured in accordance with an embodiment.

FIG. 20 depicts a Tong Ren wave transducer device configured in accordance with an embodiment.

FIG. 21 depicts a high frequency transducer device configured in accordance with an embodiment.

FIG. 22 depicts a low frequency Tong Ren wave transducer device configured in accordance with an embodiment.

FIG. 23 depicts Tong Ren wave eyeglasses device configured in accordance with an embodiment.

FIG. 24 depicts a Tong Ren wave headset device configured in accordance with an embodiment.

FIG. 25 depicts application of headset transducer device configured in accordance with an embodiment. The 25A location is for the neurological locations on the brainstem area. The 25B location is on the neck near two major arteries. The 25C location is near the thyroid with meridian points ST 9 and ST10 in the neck area. The 25D location is on the face near the gums and temporomandibular joint.

FIG. 26 depicts Tong Ren wave head band device configured in accordance with an embodiment.

FIG. 27 depicts a Tong Ren wave belt device configured in accordance with an embodiment.

FIG. 28 depicts meridian point stimulation with the use of the Tong Ren wave transducer configured in accordance with an embodiment.

FIG. 29 depicts Tong Ren brain entrainment headphone device configured in accordance with an embodiment. View 29A is side view and includes a transducer, 29B is a perspective view of headphone only.

FIG. 30 depicts a Tong Ren brain entrainment headphone device including an SD card configured in accordance with an embodiment.

### DETAILED DESCRIPTION

The invention is susceptible of many embodiments. What follows is illustrative, but not exhaustive, of the scope of the invention.

Embodiments disclose descriptions of Tong Ren brainwave recording and a brainwave to sound wave conversion process method. Tong Ren Wave applications are also disclosed in Tong Ren Wave Healing by Toni Tam, published by Oriental Healing Institute ISBN 978-0-9831023-0-4, 2012; whose contents are herein incorporated by reference. In embodiments, brainwave electroencephalography (EEG) signals are measured and recorded from a Tong Ren practitioner while he/she is conducting a Tong Ren healing session. The EEG signals of the practitioner are captured by a neural headset during the Tong Ren Healing practice. Embodiments comprise a five step process of Tong Ren brainwave recording and brainwave to sound wave conversion process. 1. Emotiv EPOC headset was used to capture and store all 14 EEG waveform output data channels. 2. All EEG signals are recorded and the EEG signals data are stored in a European Data Format (EDF) file by using Emotiv Testbench software from Emotiv Systems. 3. The stored EEG signal data in the EDF file is then processed by using custom algorithm and sound wave conversion programs. 4. Digital audio file with digital audio data was generated from step 3. 5. Digital audio file can be played by digital audio players with speakers or headphones to generate sound waves.

Various types of the brainwave generators have been developed. In these applications, most are based on the speed of the brainwave, such as the use of low frequency with Beta, Delta, or any type of brainwave frequency. They employ lowering the brainwave frequency to achieve results. For embodiments, what is notable is the form or pattern of the brainwaves. Different areas of the brain have a different frequency; also, different states of consciousness exhibit different frequencies.

Embodiments include a method and system for creating a Tong Ren brainwave entrainment (TRBE) electroacoustic composition comprising the steps of recording electroencephalogram (EEG) signals of a practitioner during a session; the session comprising the practitioner selecting at least one meridian point on a Tong Ren human figurine corresponding to a corresponding location where a subject has bioelectric blockages common to the subject's corresponding at least one specific ailment; the session further comprising the practitioner rhythmically tapping a Tong Ren hammer on the Tong Ren human figurine; the recording comprising at least one channel of the EEG signals of the practitioner; removing entire dc offset for each of the at least one channel of the EEG signals of the practitioner; scaling each of the at least one channel of the EEG signals of the practitioner; generating, for each of the at least one channel of the EEG signals of the practitioner, a sound wave recording, wherein the step of generating the sound wave recording comprises sound wave conversion software; saving, the at least one sound wave recording, into digital audio file format; selecting at least one of the saved at least one sound wave digital audio file format recordings; performing, on at least one of the selected sound wave digital audio file format recordings, additional audio signal editing comprising dc offset correction; performing, on at least one of the selected sound wave digital audio file format recordings, audio signal editing comprising volume amplification; optionally, on at least one of the selected sound wave digital audio file format recordings, signal filtering comprising low pass, band pass, band stop and high pass filtering; optionally mixing a plurality of signals; the plurality of signals comprising the selected sound file, other non brainwave sound tracks, other brain wave channel(s), and sound file(s); whereby the TRBE electroacoustic composition corresponding to the at least one specific ailment is created. Elements of the invention are presented in System, Method, and Device organization below.

### System

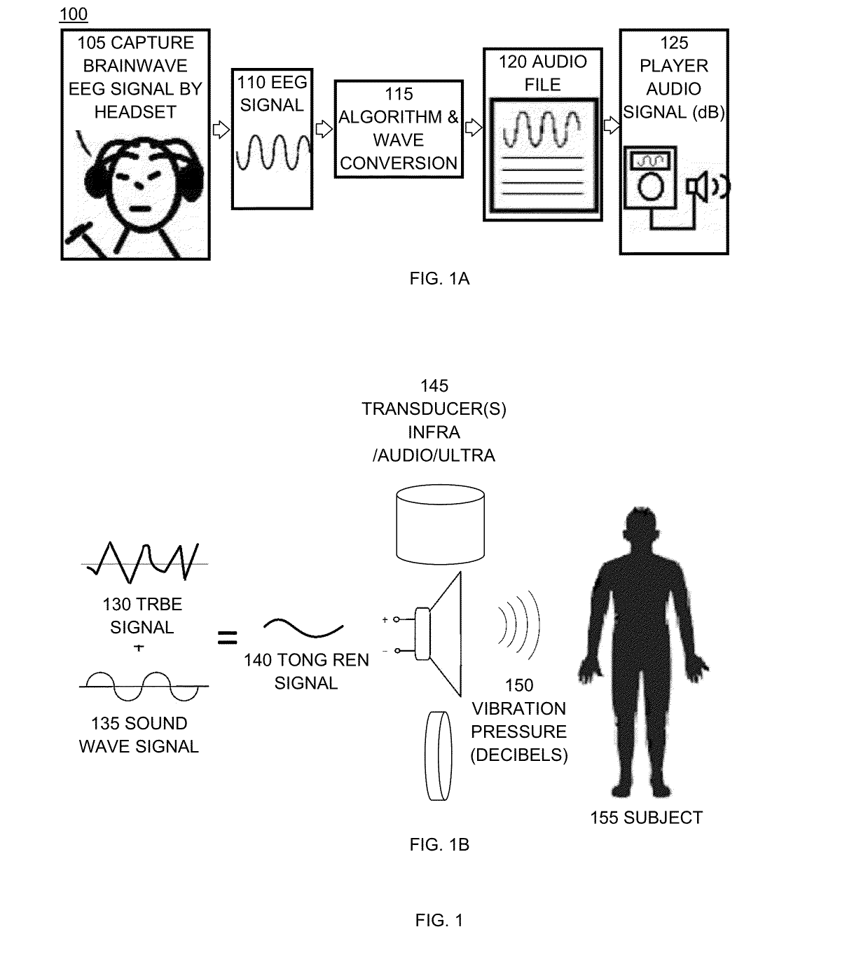

FIG. 1 depicts a block diagram 100 of a five step Tong Ren brainwave recording process 1A and overall application 1B. Tong Ren Brainwave recording process embodiments as depicted in FIG. 1A use an EEG detector to record brainwaves of the Tong Ren practitioner who is conducting a Tong Ren session 105. The resultant recorded non-transient EEG signal 110 is transformed by algorithms and wave conversion programs 115. This produces non-transient audio file 120. This file is then played, for example, in an audio/music player producing an audio signal 125. Overall application FIG. 1B depicts Tong Ren brainwave entrainment (TRBE) signal 130 which is combined with sound wave signal 135, to produce recorded non-transient Tong Ren signal 140. Recorded non-transient Tong Ren signal 140 is applied to at least one transducer 145, producing sound pressure 150 measurable in decibels to subject 155. The at least one transducer can comprise multiple transducers. These can include voice coils, infrasonic transducers, sonic transducers, and ultrasonic transducers as subsequently discussed in greater detail.

Tong Ren Brainwave Entrainment (TRBE) use is not limited to audio listening through headphones and speakers. It can be mixed with mechanical wave signals such as sound waves to create physical vibration. The mixed signal is called a Tong Ren Wave signal. This type of signal can be used to stimulate subcutaneous tissues, nerves and organs using sound wave vibration similar to ultrasound therapy machine except that it utilizes much lower frequency (from 4 hz to 200 khz), whereas ultrasound therapy machine frequencies typically extend to Mhz. Embodiments comprise broader bandwidths, including infrasound (defined as 20 Hz to 0.001 Hz). TRBE is different from Transcutaneous Electrical Nerve Stimulation (TENS) machine which uses of electric current produced by a device to stimulate the nerves for therapeutic purposes. Tong Ren wave signals does not release any electrical current to the human body, rather they produce a sequence of sound waves to create oscillation of pressure propagating through solid objects, liquid or air media. The process of using the Tong Ren wave to stimulate the body is called a Tong Ren Wave session. It is a non-invasive, using sound waves to stimulate the body.

In a typical session, the Tong Ren practitioner rhythmically taps a Tong Ren hammer on a Tong Ren human figurine, selecting meridian points on the Tong Ren human figurine where the patient has bioelectric blockages common to specific ailment(s). The use of the Tong Ren human figurine focuses the practitioner's intention and creates a link between the subconscious mind of the practitioner and points on the patient's body. Tong Ren practitioners work with patients many ways: in group class setting, one on one, by phone, or by live classes via the internet. In significant cases, working with Tong Ren more frequently speeds its impact.

In embodiments, for the last five to ten minute period of a Tong Ren session, the practitioner may also use Chi Gong techniques to enhance effects. In embodiments, the practitioner's brainwaves during this period are also recorded.

For embodiments, the brainwave recording procedure comprises devices including Emotiv EPOC neuroheadset and EPOC neuroheadset Bluetooth USB devices, and a Windows-based computer with Pentium CPU or higher. Windows® is a trademark of the Microsoft group of companies.

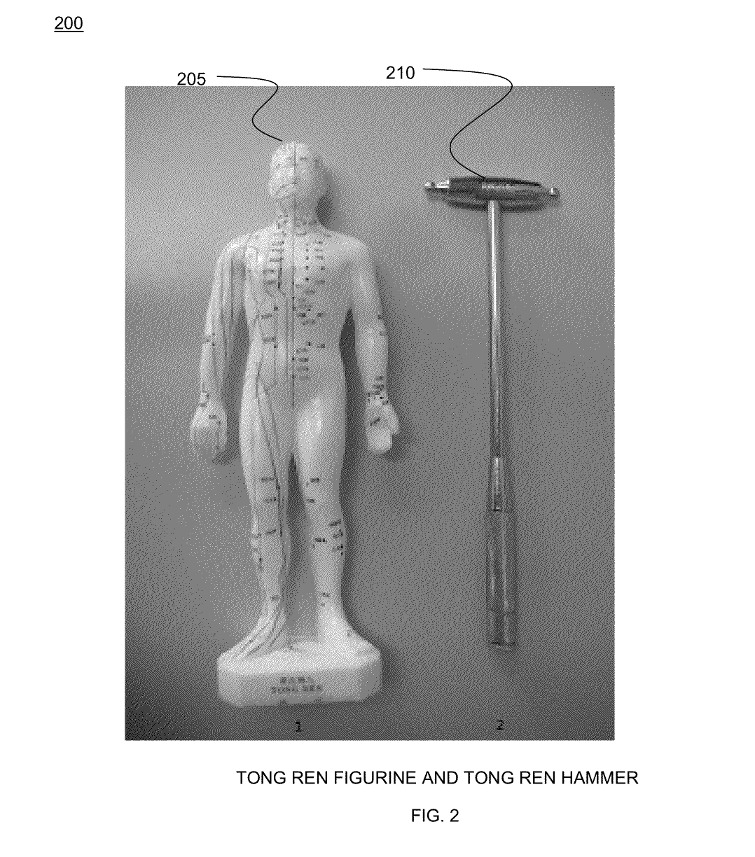

FIG. 2 depicts a Tong Ren Human Figurine employed by embodiments—a plastic, or other, model of human figure 205 marked with meridian points for reference in acupuncture training, plus a Tong Ren Tapping Hammer—a small metal, or other, hammer 210.

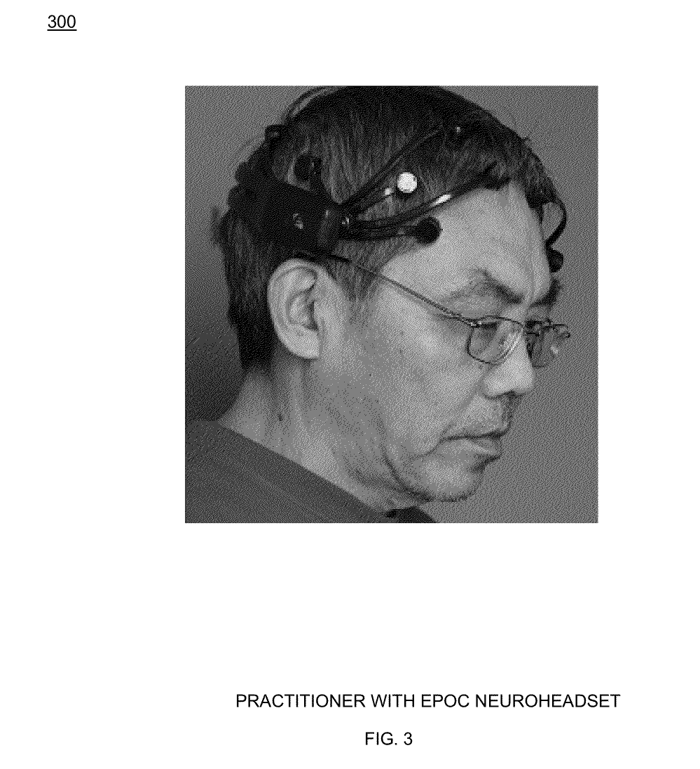

FIG. 3 displays a practitioner with a neuroheadset 300 configured in accordance with an embodiment. The neuroheadset sensors are adjusted for contact quality.

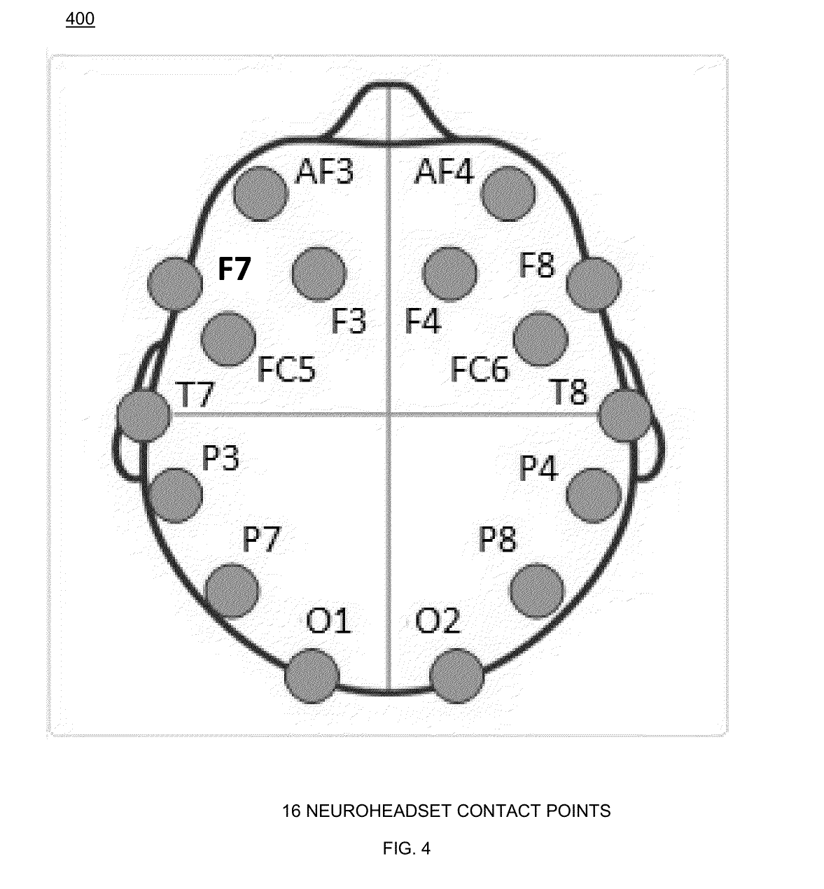

FIG. 4 shows the physical locations of all sixteen contact points 400. These should all have a green status for best contact quality and enhanced signal recordings. The sixteen contact points are designated as AF3, F7, F3, FC5, T7, P7, O1, O2, P8, T8, FC6, F4, F8, AF4, P3, and P4.

Once the Tong Ren practitioner starts the Tong Ren healing session, brainwave recording begins and the fourteen channels of EEG data AF3, F7, F3, FC5, T7, P7, O1, O2, P8, T8, FC6, F4, F8, and AF4 are recorded. Steps are also addressed in a subsequent flow chart.

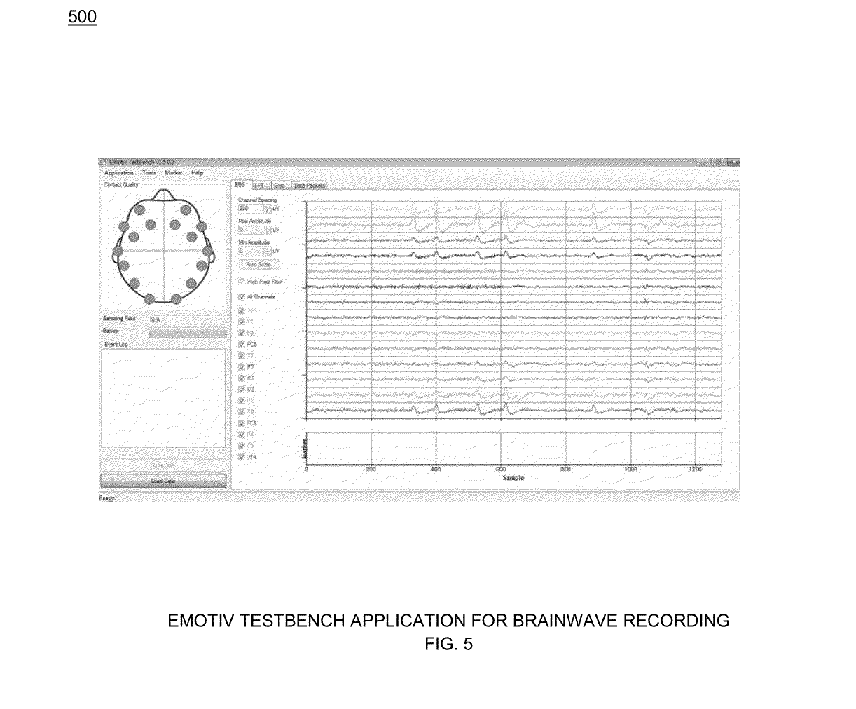

FIG. 5 depicts a screen 500 of a TestBench™ application for brainwave recording configured in accordance with an embodiment of the invention.

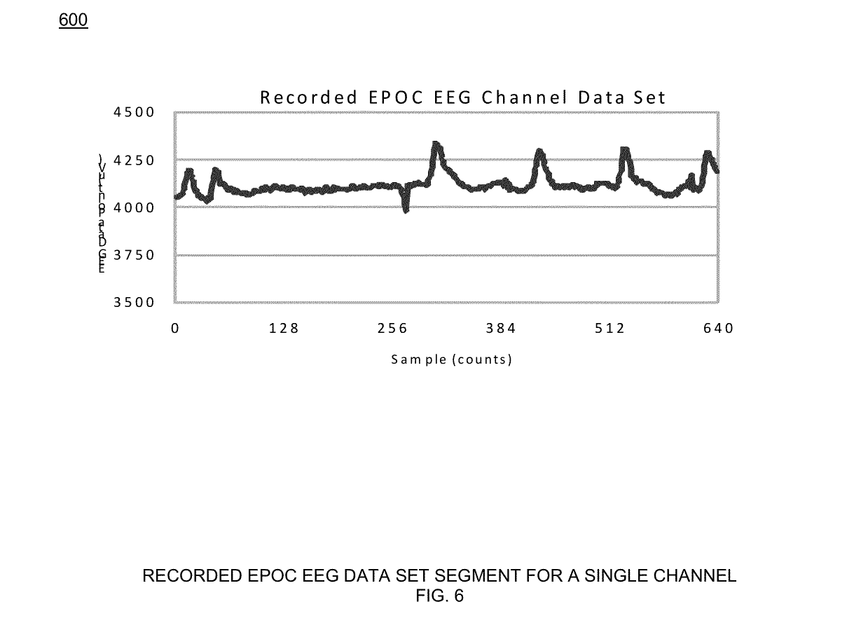

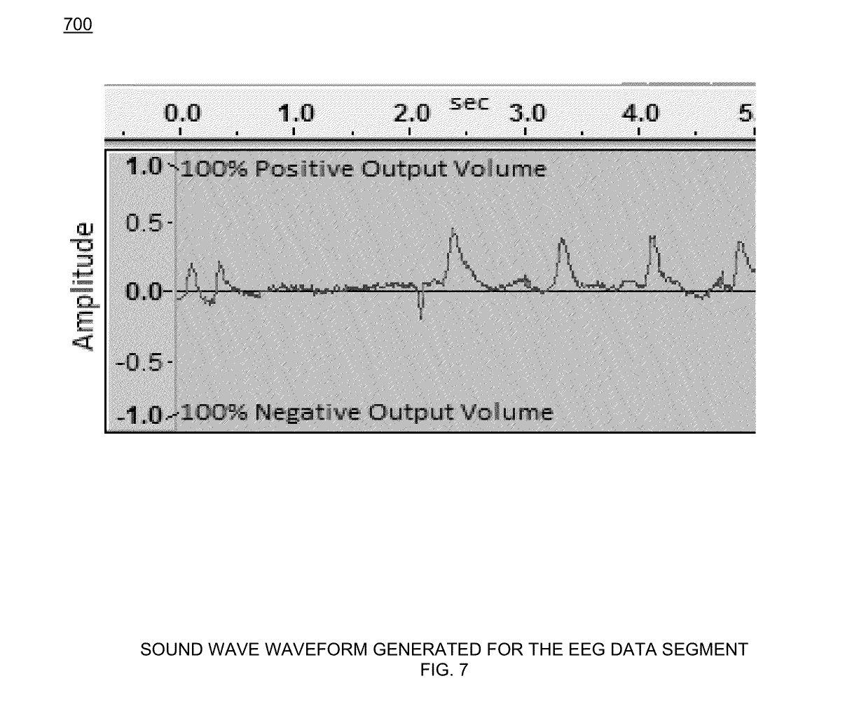

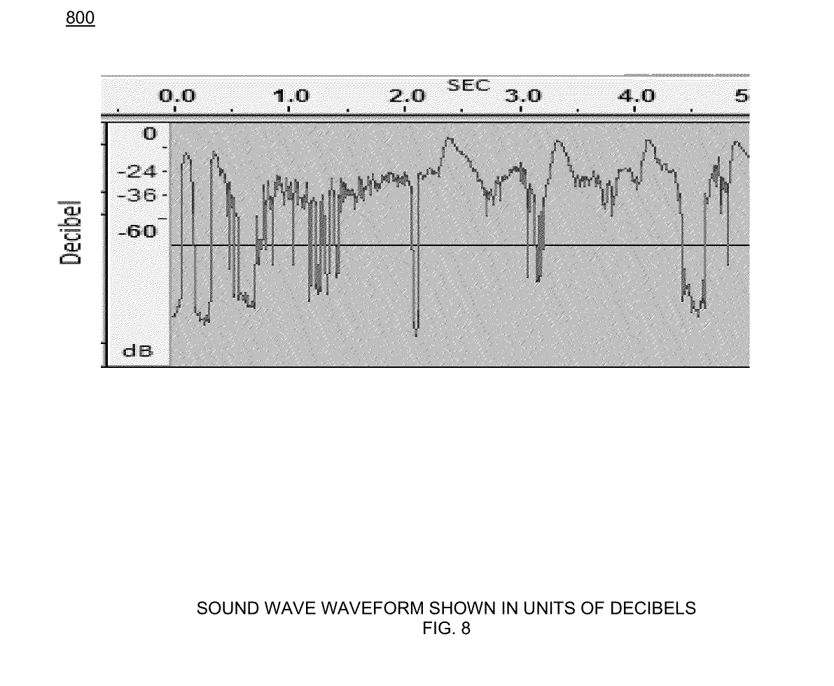

FIG. 6 depicts a graph 600 of a segment of the of the recorded EEG data stream for a single channel configured in accordance with an embodiment. The brainwave-to-sound wave conversion process transforms the recorded EEG data stream as in FIG. 6, into a sound wave as in FIG. 7.

FIG. 7 depicts a normalized graph 700 of the generated sound wave as a result of the conversion.

FIG. 8 depicts a graph 800 of the sound wave in units of decibels. Data processing algorithms and conversion software are used in this process. Steps are also addressed in a subsequent flow chart.

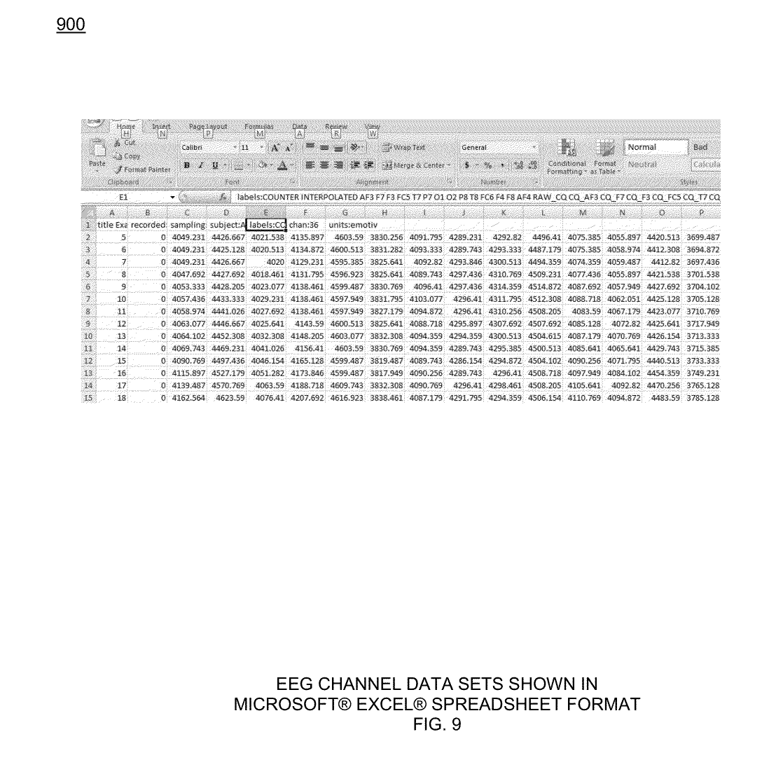

FIG. 9 depicts EEG channel data sets 900 shown in Excel® spreadsheet format configured in accordance with an embodiment. In a second conversion step the Excel® spreadsheet file (for example EEGData.xlsx) contains all fourteen EEG data channels. Each of the fourteen channel data streams is individually extracted from the Excel® spreadsheet file and converted into sound waves for the selected channel in embodiments. For embodiments, the EEGData.xlsx file also contains other brainwave recording information, but those data are not used in the conversion process. A purpose is to convert each individual channel of EEG data into an individual sound wave.

### Audio Editing

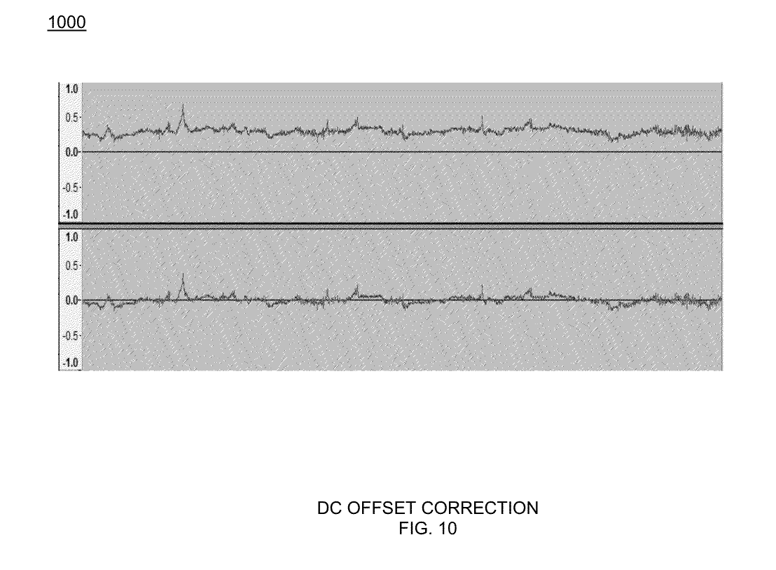

FIG. 10 depicts a graph 1000 of DC offset correction in accordance with an embodiment. In FIG. 10, the upper track is not centered on the 0.0 horizontal line and it is offset. The upper track offset is corrected by using the Normalize effect with “Remove any DC offset”. Using the Normalize function in Audacity® with this selected (with “Normalize maximum amplitude to” not selected) will correct this offset without making any changes to the peak to peak amplitudes and frequencies of the entire waveform. The corrected result will look like the lower track as shown.

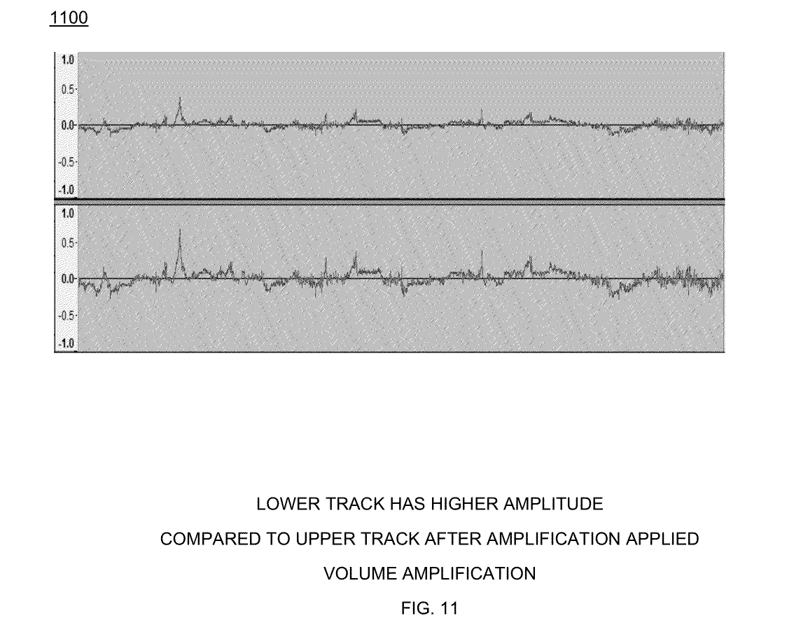

FIG. 11 depicts a graph 1100 showing a lower track with higher amplitude compared to the upper track after amplification was applied, in accordance with an embodiment of the invention.

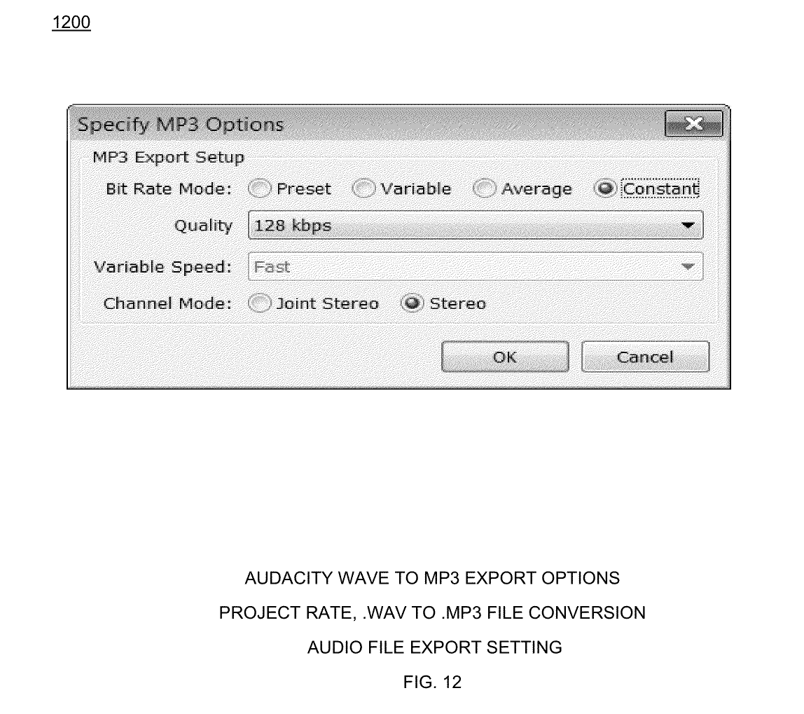

FIG. 12 depicts a screen shot 1200 of Audacity® WAVE to MP3 Export options configured in accordance with an embodiment. For embodiments, Audacity® version 1.3.12 audio editing software is utilized to perform further audio editing such as DC offset correction FIG. 10, volume amplification FIG. 11, project rate, .wav to .mp3 file conversion, audio file export setting FIG. 12. Audio editing is not limited to, but embodiments may include, signal filtering techniques such as separating frequency components from the sound wave by employing a series of low pass, band pass, band stop and high pass filters by using Audacity®, MATLAB® or other audio editing exiting software.

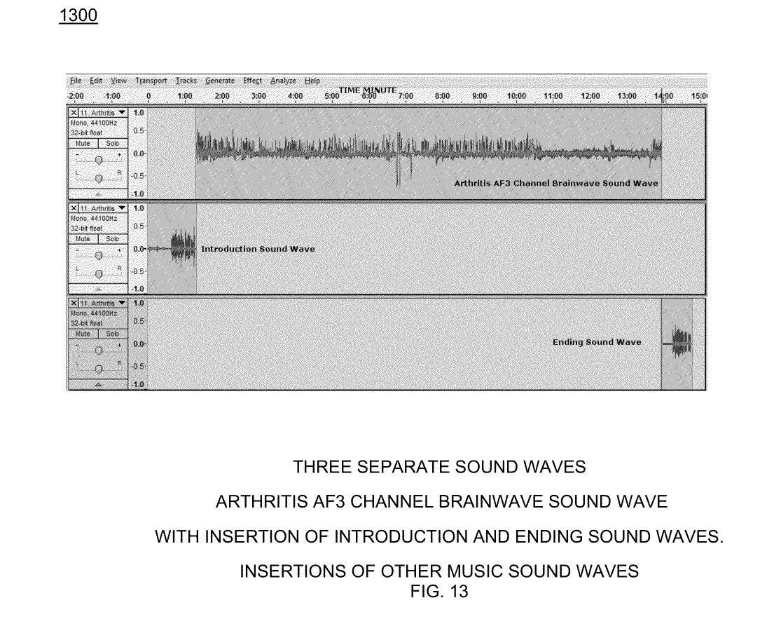

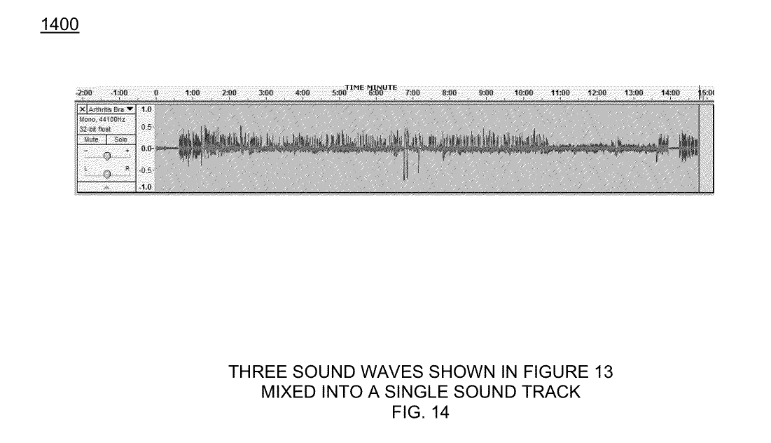

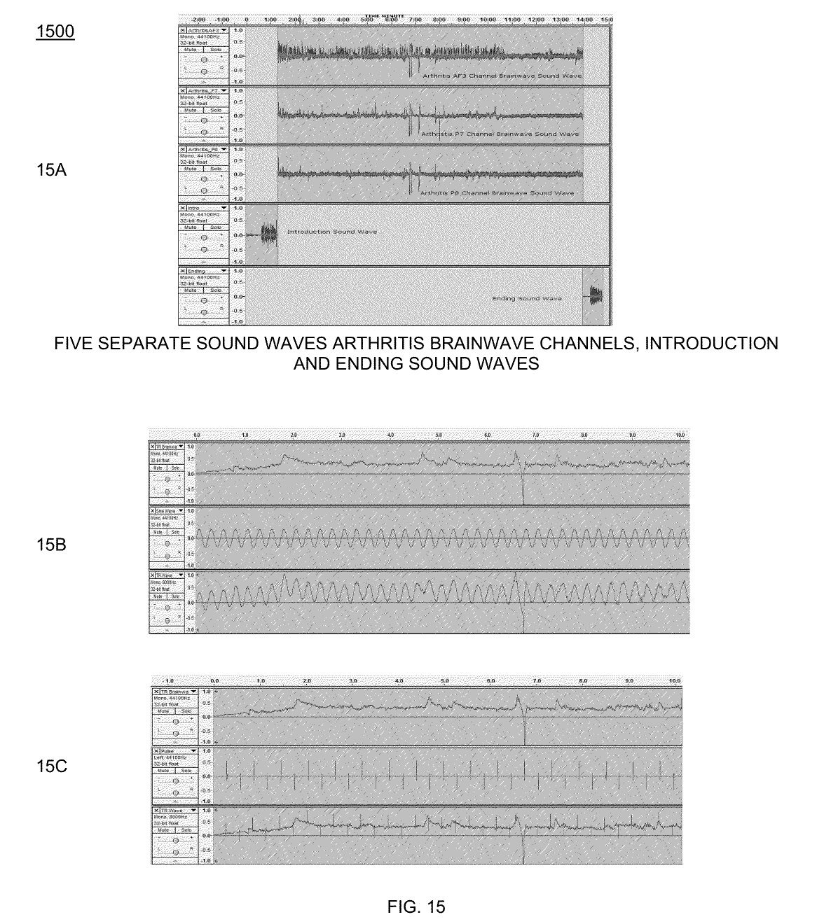

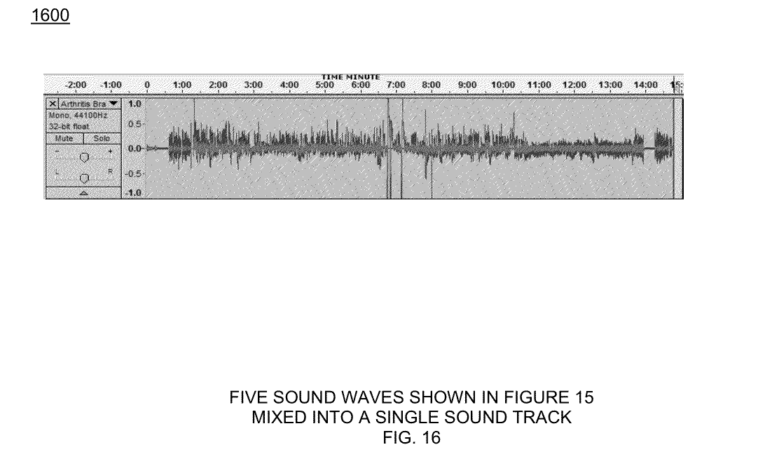

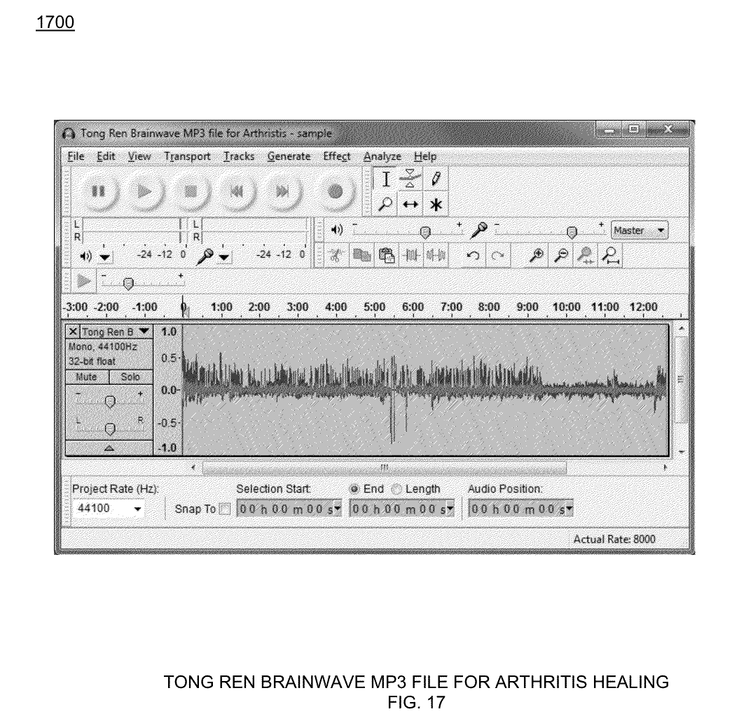

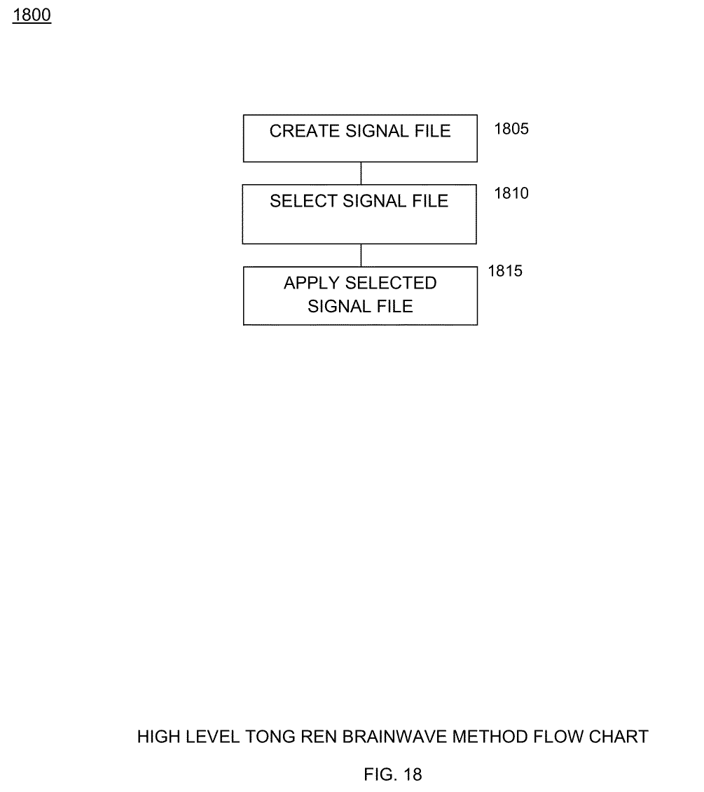

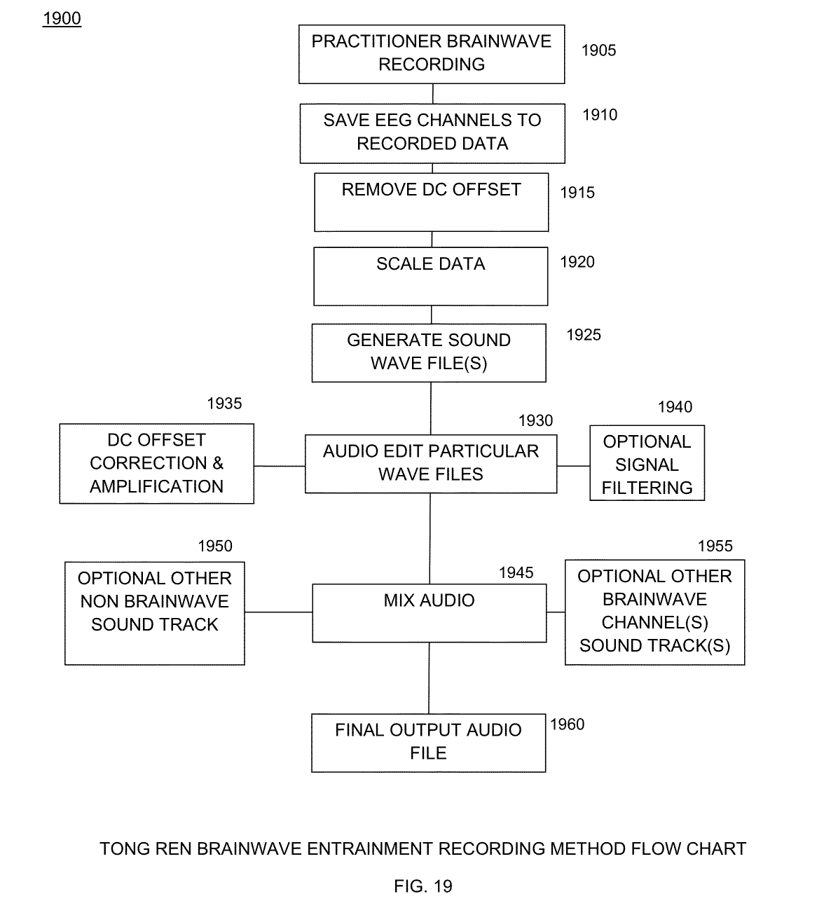

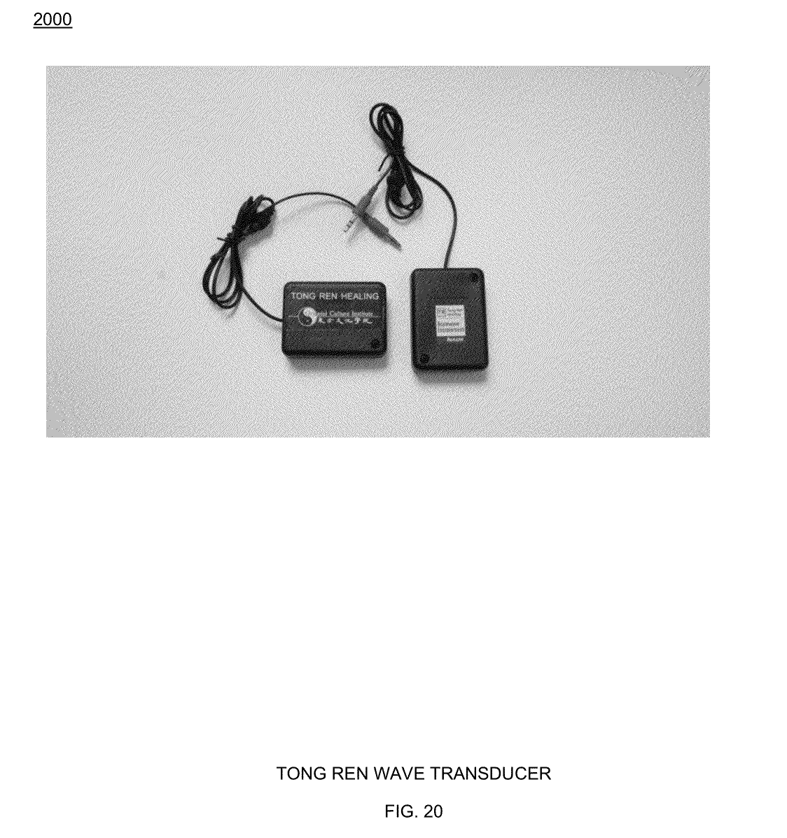

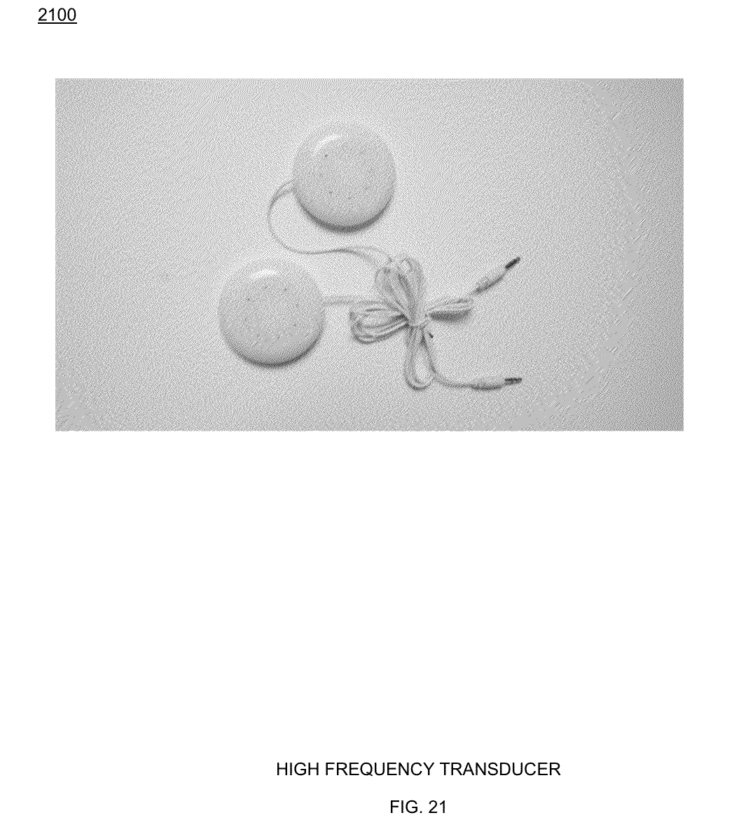

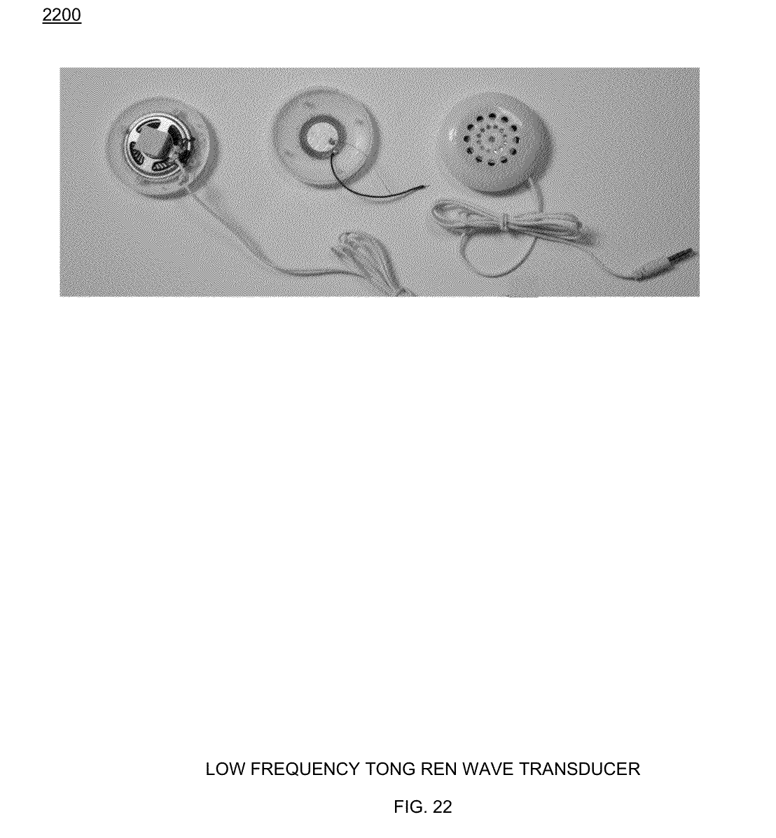

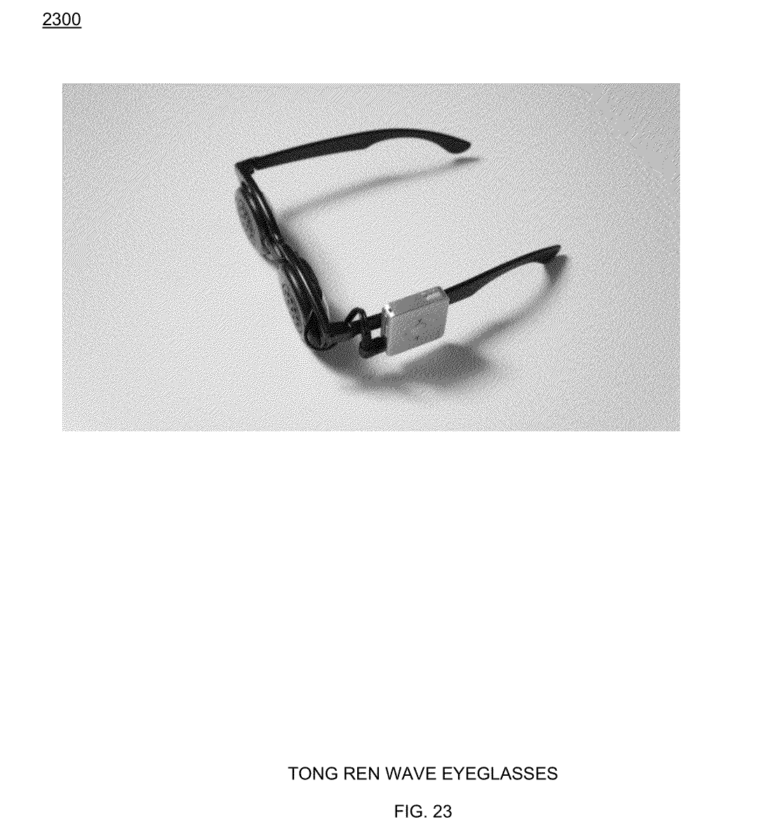

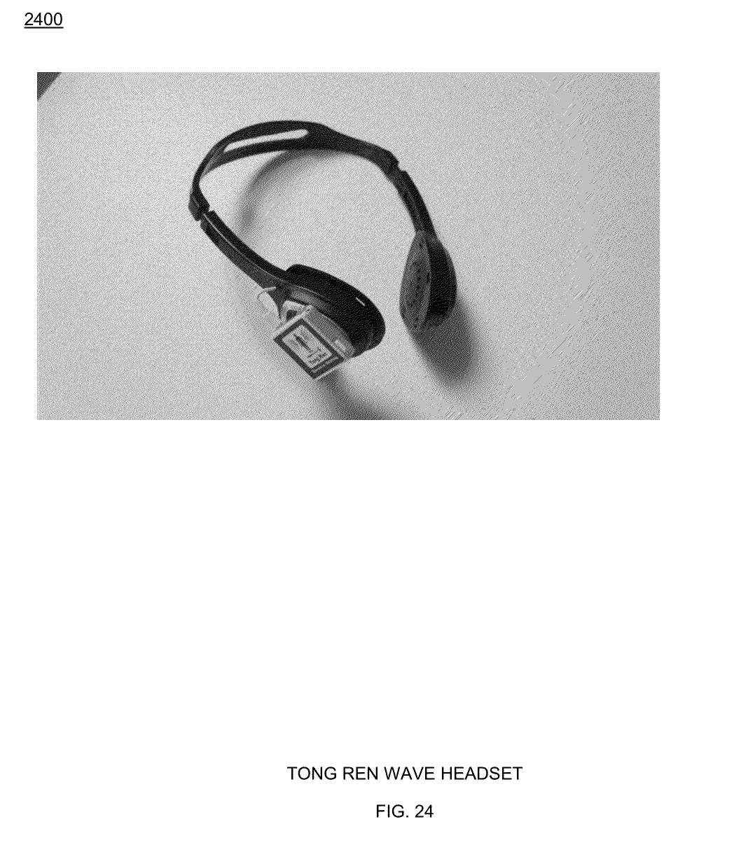

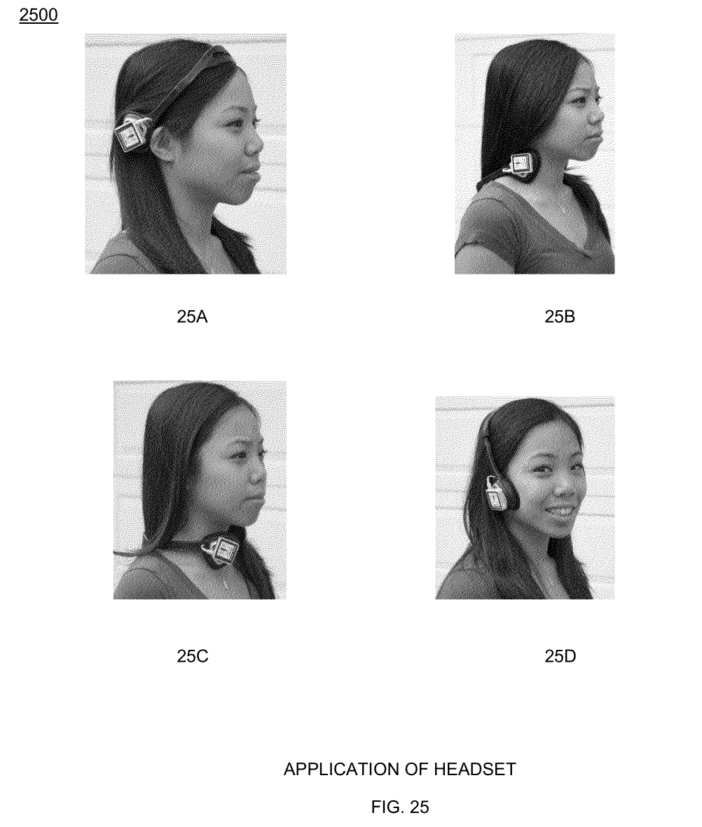

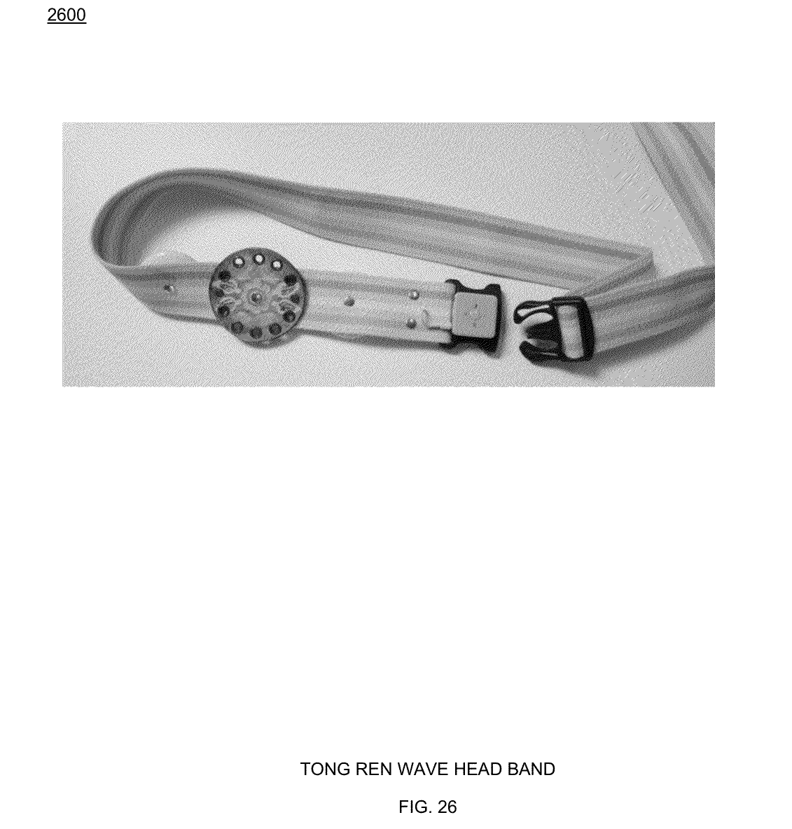

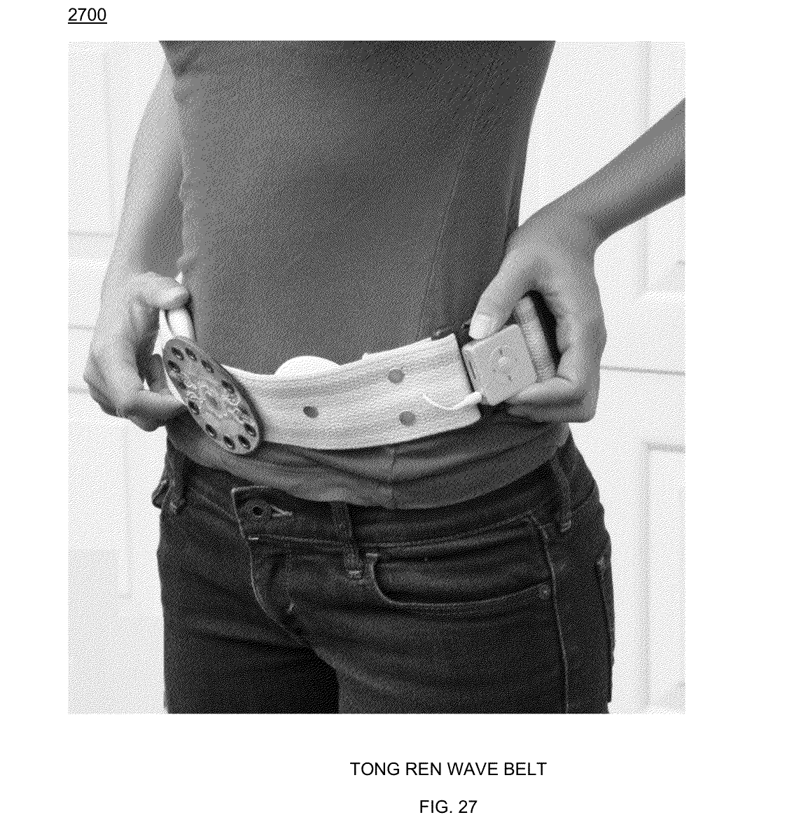

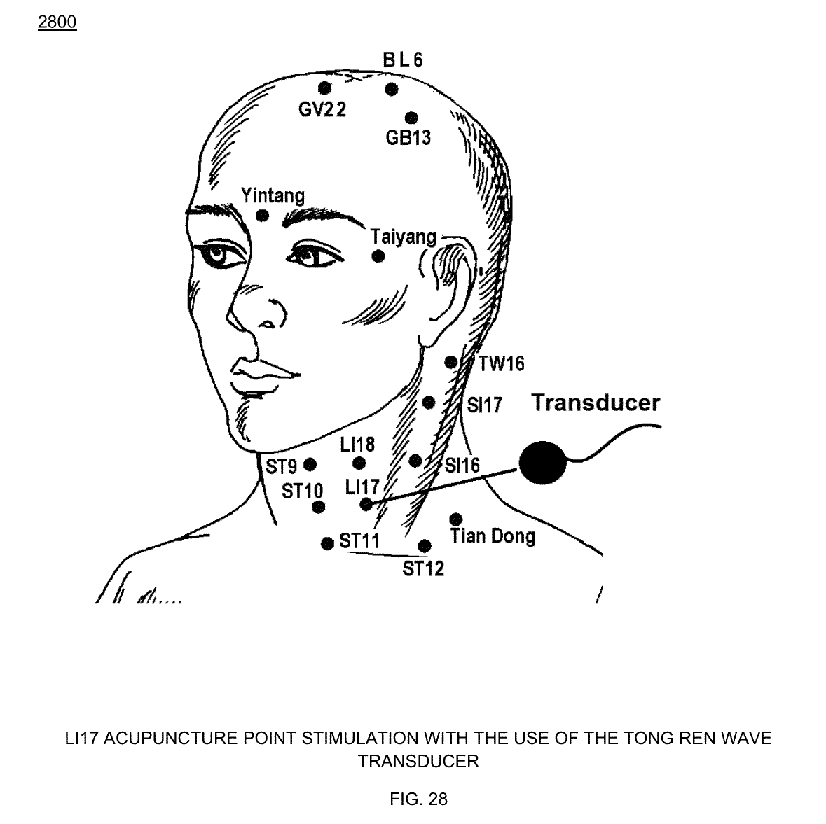

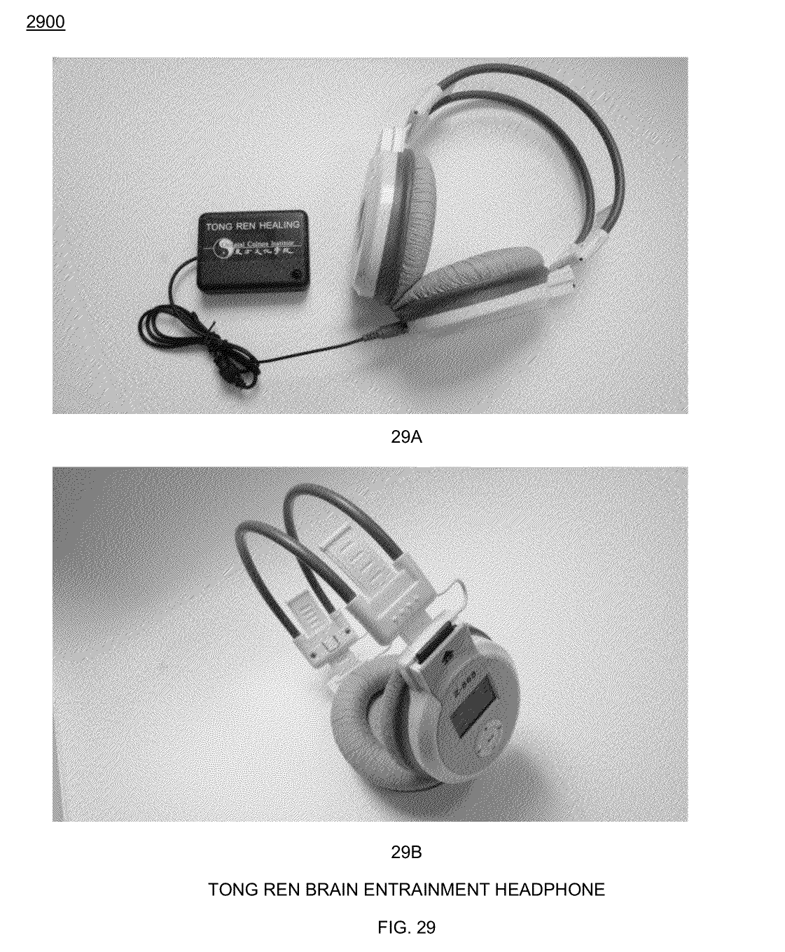

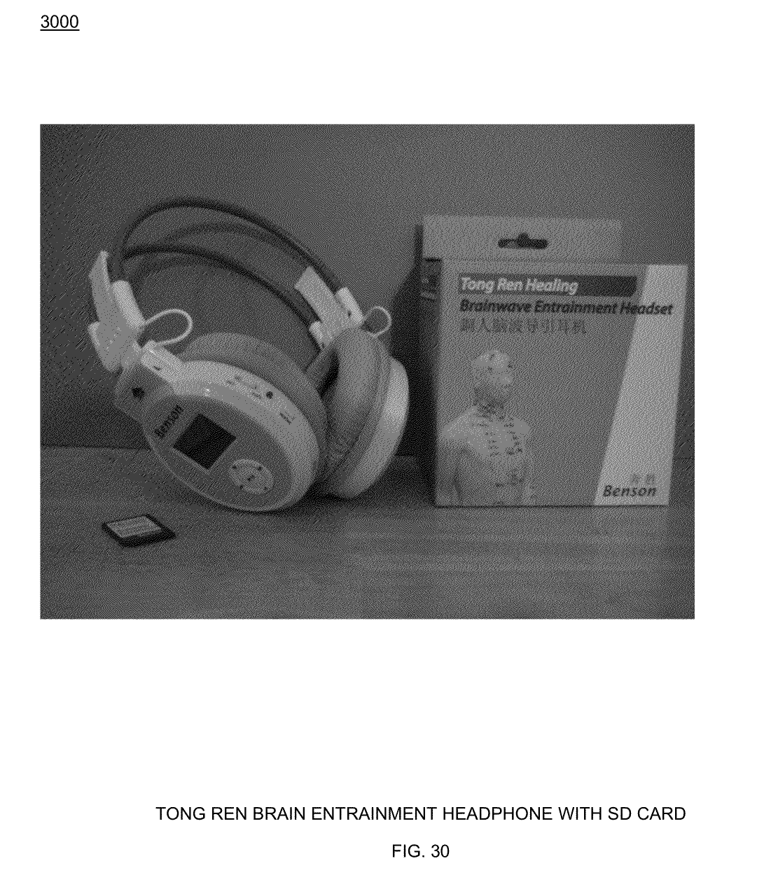

FIG. 13 depicts a graph 1300 of three separate sound waves (1) Arthritis AF3 channel brainwave sound wave with insertions of (2) introduction and (3) ending sound waves configured in accordance with an embodiment.

FIG. 14 depicts a graph 1400 of the three sound waves shown in FIG. 13 mixed into a single sound track configured in accordance with an embodiment. An audio mixing option can also be employed such as insertions of other music sound waves shown in FIG. 13 and FIG. 14, or combined with other brainwave channel sound waves to existing wave files as desired (FIGS. 15 and 16).

FIG. 15 depicts graphs 1500 of 15A five separate sound waves: Arthritis brainwave channels AF3, F7, P8, introduction and ending sound waves configured in accordance with an embodiment. These sound waves can include, but are not limited to music, human speech and other sound recordings. Graph 15B depicts mixing a 4 Hz sine wave with a TRBE audio file. Graph 15C depicts mixing a pulse wave with a TRBE audio file. The process of creating a Tong Ren wave file comprises mixing a TRBE audio file and a sound wave file together similar to the five separate sound waves of 15A. Various sound waves can be mixed with the TRBE audio file. Most commonly use sound wave waveforms for the mixing process are sine waves and pulse waves. At the current stage, the sound wave frequency for the Tong Ren wave file is in the range between 4 Hz and 200 kHz. This does limited Tong Ren wave to be mixed with sound wave frequency outside of that range.

FIG. 16 depicts a graph 1600 of all five sound waves shown in FIG. 15 mixed into a single sound track configured in accordance with an embodiment.

FIG. 17 depicts a display 1700 of a Tong Ren Brainwave MP3 file configured in accordance with an embodiment. Embodiments address arthritis mitigation.

### Method

FIG. 18 is a high-level flow chart 1800 of a method for Tong Ren Brainwave Entrainment in accordance with an embodiment of the invention. Signal files are created 1805 by recording and processing signals during a session identified with at least one indication. One of these files is selected for application 1810. The selected files is then applied 1815.

FIG. 19 is a flow chart of the recording creation operation of a method of Tong Ren Brainwave Entrainment 1900 configured in accordance with an embodiment. This step corresponds to step 1805 of FIG. 18. At start, all fourteen EEG Channel signals of the practitioner are recorded during Tong Ren session 1905. The fourteen EEG Channel recorded EEG signals are saved in computer readable file 1910. For each channel, DC offset is removed entirely 1915. For each channel, signal scaling process is applied 1920. For each channel, a sound wave is generated and saved into digital audio file format 1925 by using sound wave conversion software. Any particular one of the fourteen individual sound wave files can be chosen to perform additional audio signal editing 1930. This includes DC offset correction and volume amplification 1940, and optional signal filtering 1940 such as low pass, band pass, band stop and high pass filters. Optional audio mixing 1945 can also be implemented for the selected sound file with other non brainwave sound tracks 1950 and/or with other brain wave channel(s) sound file(s) 1955. Final audio file output is complete 1960.

Additional elaboration on these steps is provided next. Embodiments employ the Emotiv TestBench™ software application. In embodiments, the recording and device setup comprises: 1) Power on neuroheadset, ensuring headset is fully charged. 2) Plug in the neuroheadset Bluetooth device into a computer USB port. This Bluetooth device allows wireless data communication between the neuroheadset and the computer for brainwave recording. The practitioner freedom of movement derived from this enables enhanced recorded signals by reducing constraints and distractions. 3) Load and start TestBench™ software. In embodiments, TestBench™ software is used for recording brainwave signals from the neuroheadset. Software can display EEG waveform recordings in real time. For embodiments, EEG data are recorded in units of microvolts (μV) and stored in European Data Format (EDF) files. 4) Locate EPOC neuroheadset on Tong Ren Practitioner scalp. For the next step, 5) Adjust neuroheadset sensors for contact quality. For the next step, 6), once the Tong Ren practitioner starts the Tong Ren healing session, brainwave recording begins by pressing the Save Data button in the TestBench™ Application. Fourteen channels of EEG data AF3, F7, F3, FC5, T7, P7, O1, O2, P8, T8, FC6, F4, F8, and AF4 are recorded in real time with sample rate of 128 samples per second, in embodiments. Steps are also addressed in a subsequent flow chart. Real-time display of the headset data streams for all fourteen channels, and is displayed on the screen for embodiments, giving the practitioner real-time visual feedback. This real-time visual feedback supports adaptive tapping wherein the practitioner changes tapping intensity and/or rate to increase signal amplitude. For step 7), when the Tong Ren healing practice session is completed, brainwave recording can be stopped by pressing the Stop Saving button in the TestBench™ Application. All fourteen channels of Brainwave EEG data points are stored in an EDF file. For embodiments, this EDF file is used next in the brainwave-to-sound wave conversion process. Steps are also addressed in a subsequent flow chart. Note that the TestBench Application does not record P3 and P4 channels. In embodiments, the EPOC headset utilizes P3 and P4 for CMS/DRL references and no measurements are made to P3 and P4 channels.

The conversion process converts any of the selected single EEG channel recorded data streams into a sound wave stream which is stored in Waveform Audio File (WAV) format in embodiments. For example, assume the AF3 channel is selected for conversion. After the AF3 channel is converted into a sound wave, the digital audio file that was generated by the conversion process is named AF3.way. For embodiments, this conversion process is applied to all 14 channels' stream data and generates 14 individual WAV files, one for each channel. For embodiments, a user can select fewer than 14 channels to convert into sound waves.

In embodiments, the computer processing equipment includes hardware comprising a Windows based computer with a Pentium CPU or higher. Software includes the Emotiv TestBench™ Application; Microsoft Excel® 2007 or later version; MATLAB® software (similar software such as QtOctave also acceptable for embodiments); and Audacity® audio editing software. MATLAB® is a registered trademark of The MathWorks, Inc. Audacity® is a registered trademark of Dominic M. Mazzoni of Fremont Calif.

Embodiments of the conversion process comprise the following steps. Conversion process step 1) convert the EDF file format to a CSV file, in embodiments by using TestBench™ Software—EDF to CSV convertor tool; then use Microsoft® Excel® spreadsheet to open the CSV and save the file as Excel® .xlsx format. In a second conversion step, 2), the Excel® spreadsheet file (for example EEGData.xlsx) contains all fourteen EEG data channels, namely AF3, F7, F3, FC5, T7, P7, O1, O2, P8, T8, FC6, F4, F8, AF4. Each of the fourteen channel data streams is individually extracted from the Excel® spreadsheet file and converted into sound waves for the selected channel in embodiments. For embodiments, the EEGData.xlsx file also contains other brainwave recording information, but those data are not used in the conversion process. A purpose is to convert each individual channel of EEG data into an individual sound. wave. In conversion step 3), the algorithm is applied for data processing for each EEG channel data stream for the data to be used in the next sound wave conversion process step.

The steps of the algorithm are detailed as follows: a) Remove DC offset from the EEG data stream, and b) Scale the EEG data stream with DC offset removed to have value within between −1 and +1. In conversion step 4), EPOC EEG data is converted into sound wave data involving several operations. First, DC offset is removed from all EEG data points. All EEG data point measurements contain approximately a 4,000 μV DC offset. This DC offset is embedded by EPOC. A DC offset removal algorithm is applied to each data point of the channel data set.

An embodiment of the DC offset removal algorithm functions as follows: Channel Data Points DC offset removed (μV)=Channel Data Points (μV)−Channel Data Middle Point (μV). Channel Data Points with DC offset removed are the data with DC offset value subtracted. Channel data points are the original EEG data recorded data with DC offset.

The channel middle point or midpoint (Mid) is a constant real number which indicates the half way point between the maximum (Max) value and the minimum (Min) value of the entire channel data set. The channel Max point is the highest value of entire channel data set. The channel Min point is the lowest value of entire channel data set.

The formula for finding the channel Mid point is: Channel Mid Point (μV)=[((Channel Max Point (μV)−Channel Min Point (μV)/2)+Channel Min Point (μV)]

For example: Given an AF3 EEG channel data set with 4 data points−Channel AF3 (μV)=[3900, 4100, 3800, 4220]; Channel Mid Point is ((4220 μV−3800 μV)/2)+3800 μV=4010 μV. Subtract 4010 μV from each data point to remove the DC offset. AF3DC offset removed (μV)=[(3900−4010), (4100−4010), (3800−4010], (4220−4010)]. AF3 DC offset removed (μV)=[−110, 90, −210, 210], this resultant data set is the AF3 processed data set after DC offset removed.

In conversion step 5), after the DC offset is removed from the data set, the signal scaling process is performed to scale all data in the AF3 channel data set to be within a −1 to +1 range. For embodiments, signal scaling, which is similar to an audio normalization technique, is applied to the entire channel by increasing (or decreasing) the amplitude of an entire signal so that the resulting peak amplitude matches a desired target.

The signal scaling formula is as follow: Scaled Data=Channel Data Points with DC offset removed/Peak value. Peak value is determined by the highest data point in the data set; all data points will be treated as absolute values.

For example: Absolute values of AF3 (μV) are [110, 90, 210, 210]. AF3 peak value is=210 (μV). Next, all AF3 data points are divided by AF3 peak value as follows: AF3scaled (μV)=[(−110/210), (90/210), (−210/210), (210/210)]. AF3scaled (μV)=[−0.5238, 0.4286, −1, 1], this resultant data set is the AF3 processed data set after the scaling process.

Conversion Software

For embodiments, MATLAB® software is utilized for sound wave conversion. MATLAB®'s wave write function is used for this conversion process. MATLAB®'s wave write function will write each of the EEG processed data points into sound units of decibels (dB)s and store the decibel value into a newly generated 16 bit or 32 bit waveform audio file (for example, AF3.wav). This wave file can be read by personal computers with digital audio software or other digital audio players.

For example: Wave Write Function syntax−wavwrite (Y, Fs, N, filename). Y=amplitude input data between −1 to +1, Fs=Sample Rate, N=Bits, filename=specify output file name. When wavwrite (AF3 processed data set, 128, 32, ‘AF3.wav’) function command is executed, AF3.wav (a 32 bit WAVE file) is generated by MATLAB® for the AF3 channel.

For embodiments, the previous DC offset and scaling processes were employed because the wavwrite function only accepts amplitude data input range between −1 and +1. Otherwise, the wavwrite function will not execute properly.

The Tong Ren Brainwave entrainment technique can be used by phone for long distance healing. This technique applies to use of a digital audio player such as personal computers, MP3, MP4 music players, and CD players, which require very little technical training. Embodiments of this method can be applied to animals.

### Devices

FIG. 20 depicts a Tong Ren wave transducer device 2000. In embodiments, the Tong Ren wave transducer for transmitting the Tong Ren wave signals via sound wave to the human body is made up of voice coil actuator such as audio speaker. The Tong Ren wave transducer is non-invasive to the body skin or tissues. Its function produces a sequence of sound wave pressure to the body for stimulation. The Tong Ren Wave signal can be applied to piezoelectric material type transducers similar to the transducer probe used in ultrasound machines. For Example, Tong Ren Wave also can be play by a vibration transducer/speaker to produce physical vibration for stimulation and healing effect. Users place the vibration part of the device on the surface to start a Tong Ren Wave session. Vibration speakers such as the portable vibration speaker ROCK-IT 3.0 produced by OrigAudio can be used for sessions in embodiments.

FIG. 21 depicts a high frequency transducer device 2100 operating from 20 kHz to 200 kHz for Tong Ren wave sessions. This transducer can be connected to an audio player and placed on top of the skin for stimulation and other effects.

FIG. 22 depicts a low frequency Tong Ren wave transducer device 2200 operating from 4 Hz to 20 kHz for Tong Ren wave sessions. This transducer can be connected to an audio player and placed on body surface for stimulation. A bass speaker for low frequency (left side) and a tweeter speaker for high frequency (middle) together form the Tong Ren Wave transducer (right side). Tong Ren Waves can be applied without placing the transducer on top of the skin. The Tong Ren Wave can also be played by commercially available audio speakers away from the human body, since the sound waves propagate via air medium to the human body for stimulation and other effects.

FIG. 23 depicts Tong Ren wave eyeglasses device 2300 with two transducers to produce and direct the Tong Ren wave signal to stimulate the eye area. For embodiments, the audio player is integrated to the eyeglasses.

FIG. 24 depicts a Tong Ren wave headset device 2400 configured in accordance with an embodiment.

FIGS. 25A-D depict applications of the headset transducer device 2500. The 25A location is for the neurological locations on the brainstem area. The 25B location is on the neck near two major arteries. The 25C location is near the thyroid with meridian points ST 9 and ST10 in the neck area. The 25D location is on the face near the gums and temporomandibular joint.

FIG. 26 depicts Tong Ren wave head band device 2600.

FIG. 27 depicts a Tong Ren wave belt device 2700 configured in accordance with an embodiment. Transducers in Figure 2100 are attached to the belt so that user can wear it around the stomach area for stimulation using the Tong Ren wave. The belt can be turned around to have the transducers facing the back area to for back stimulation.

FIG. 28 depicts LI17 meridian point stimulation 2800 with the use of the Tong Ren wave transducer to stimulate meridian points. The transducer can be used for stimulating specific acupuncture points without the use of acupuncture needles by placing the transducer on the meridian point surface for stimulation. For example, using Tong Ren Waves to stimulate the LI17 meridian point: the transducer is placed on the LI17 surface area shown.

FIG. 29 depicts Tong Ren brain entrainment headphone device 2900. The Tong Ren Wave Transducer and Tong Ren Brain Entrainment Headphone can be used simultaneously by connecting the transducer to the output the of the headphone. The user can listen to the sound wave and use the transducer for physical stimulation at the same time. For embodiments, the user uses the TRBE headset to listen to the TRBE audio file. The headset allows the user to insert and play the TRBE audio files which were previously stored in the SD card.

FIG. 30 depicts Tong Ren brain entrainment headphone device 3000 including an SD card.

As mentioned, additional embodiments comprise vibration transducers or vibration speakers in producing output. Nonlimiting producers of vibration speakers include Rock-It by OrigAudio, GigaCube by Gigabiz Ltd, and Vibe IT by Wide Ideas Inc.

Tong Ren Wave device use comprises: connecting the Tong Ren Wave transducer to the audio output of an audio/music player; activating the Tong Ren Wave transducer by playing the Tong Ren Wave file (MP3 File) from the audio/music player; placing the Tong Ren Wave transducer against the surface of the body of the specific area of the body to be stimulated. As the transducer vibrates, it delivers sound wave through the body into deep tissues, nerves, and organs of the body. If headphone audio player is available, user can listen to the Tong Ren Wave and active the transducer at the same time if found applicable.

The foregoing description of the embodiments of the invention has been presented for the purposes of illustration and description. Each and every page of this submission, and all contents thereon, however characterized, identified, or numbered, is considered a substantive part of this application for all purposes, irrespective of form or placement within the application. This specification is not intended to be exhaustive or to limit the invention to the precise form disclosed. Many modifications and variations are possible in light of this disclosure. Other and various embodiments will be readily apparent to those skilled in the art, from this description, figures, and the claims that follow. It is intended that the scope of the invention be limited not by this detailed description, but rather by the claims appended hereto.

I claim:

1. A system for creating a Tong Ren brainwave entrainment electroacoustic (TRBE) composition comprising the steps of:
recording electroencephalogram (EEG) signals of a practitioner during a session;
said recording comprising at least one channel of said EEG signals of said practitioner;
removing DC offset for each of said at least one channel of said EEG signals of said practitioner;
scaling each of said at least one channel of said EEG signals of said practitioner;
generating, for each of said at least one channel of said EEG signals of said practitioner a sound wave recording;
saving said at least one sound wave recording;
selecting at least one of said saved at least one sound wave recordings;
performing, on at least one of said selected sound wave recordings, additional audio signal editing comprising DC offset correction; and
performing, on at least one of said selected sound wave recordings, audio signal editing comprising volume amplification;
whereby said TRBE electroacoustic composition corresponding to said at least one specific ailment is created.

2. The system of claim 1, wherein said recording of at least one channel of said EEG signals of said practitioner comprises fourteen channels.

3. The system of claim 1, wherein said session further comprises said practitioner rhythmically tapping a Tong Ren hammer on a Tong Ren Human figurine.

4. The system of claim 1, wherein said at least one sound wave recording is in digital audio file format.

5. The system of claim 1, further comprising:
selecting a recorded session composition;
connecting a Tong Ren Wave transducer to audio output of an audio/music player;
activating said Tong Ren Wave transducer by playing said selected Tong Ren Wave file from said audio/music player; and
placing said Tong Ren Wave transducer against surface of body of specific area to be stimulated.

6. The system of claim 5, comprising:
bone conduction of said transmission.

7. The system of claim 1, comprising:
said transmission comprising about 25 kilohertz at about 10 milliwatts power.

8. The system of claim 5, wherein said transmission is between about 4 Hz to about 200 kHz.

9. The system of claim 5 wherein said step of placing said Tong Ren Wave transducer is accomplished remotely over communication lines from source of said transmission.

10. The system of claim 5, further comprising:
a low frequency Tong Ren wave transducer device.

11. The system of claim 5, further comprising:
a high frequency Tong Ren wave transducer device.

12. The system of claim 5, further comprising:
a Tong Ren wave eyeglasses device.

13. The system of claim 5, further comprising:
a Tong Ren wave head band device.

14. The system of claim 5, further comprising:
a Tong Ren wave belt device.

15. A method for creating a Tong Ren brainwave entrainment (TRBE) electroacoustic composition comprising the steps of:
recording electroencephalogram (EEG) signals of a practitioner during a session;
said session comprising said practitioner selecting at least one meridian point on a Tong Ren Human figurine corresponding to a corresponding location where a subject has bioelectric blockages common to said subject's corresponding at least one specific ailment;
said session further comprising said practitioner rhythmically tapping a Tong Ren hammer on said Tong Ren Human figurine;
said recording comprising at least one channel of said EEG signals of said practitioner;
removing entire DC offset for each of said at least one channel of said EEG signals of said practitioner;
scaling each of said at least one channel of said EEG signals of said practitioner;
generating, for each of said at least one channel of said EEG signals of said practitioner, a sound wave recording, wherein said step of generating said sound wave recording comprises sound wave conversion software;
saving said at least one sound wave recording into digital audio file format;
selecting at least one of said saved at least one sound wave digital audio file format recordings;
performing, on at least one of said selected sound wave digital audio file format recordings, additional audio signal editing comprising DC offset correction; and
performing, on at least one of said selected sound wave digital audio file format recordings, audio signal editing comprising volume amplification;
whereby said TRBE electroacoustic composition corresponding to said at least one specific ailment is created.

16. The method of claim 15, comprising:
accomplishing, on at least one of said selected sound wave digital audio file format recordings, signal filtering comprising low pass, band pass, band stop, and high pass filtering.

17. The method of claim 15, comprising:
mixing a plurality of signals.

18. The method of claim 15, wherein said plurality of signals comprises said selected sound file, other non brainwave sound tracks, at least one other brain wave channel, and at least one additional sound file.

19. A system for Tong Ren brainwave entrainment (TRBE) comprising:
recording and processing electroencephalogram (EEG) signals of a practitioner during a session;
selecting a recorded session;
connecting a Tong Ren Wave transducer to audio output of an audio/music player;
activating said Tong Ren Wave transducer by playing said selected Tong Ren Wave file from said audio/music player;
placing said Tong Ren Wave transducer against surface of body of specific area to be stimulated.

20. The system of claim 19, further comprising:
simultaneously activating a headphone audio player to listen to said Tong Ren Wave.
